(19) United States
(12) Patent Application Publication
(10) Pub. No.: US 2018/0078665 A1
(43) Pub. Date: Mar. 22, 2018

(54) MEDICAL PACKAGE

(71) Applicant: Covidien LP, Mansfield, MA (US)

(72) Inventor: James Buccellato, Blaine, MN (US)

(21) Appl. No.: 15/709,256

(22) Filed: Sep. 19, 2017

Related U.S. Application Data
(60) Provisional application No. 62/396,654, filed on Sep. 19, 2016.

(51) Int. Cl. A61L 2/28; A61L 2/04; B65B 51/10; B65B 55/14; B65D 75/30; B65D 77/20; B65D 75/52; A61B 50/33

(52) U.S. Cl. CPC A61L 2/28; A61L 2/04; B65B 51/10; B65B 55/14; A61B 2050/005; B65D 77/2024; B65D 75/52; A61B 50/33; A61L 2202/24; B65D 75/305

(57) ABSTRACT

An assembly includes a medical package defining a housing configured to receive a medical supply. The medical package comprises a first sealable region and a second sealable region. The first and second sealable regions are configured to be sealed together to secure the medical supply within the housing. The medical package includes a thermal pattern configured to exhibit a first visible indicium indicative of an unsealed region between the first and second sealable regions. The thermal pattern is configured to irreversibly transform to a second visible indicium indicative of a sealed region between the first and second sealable regions in response to exposure to a temperature greater than or equal to a first temperature threshold, and to a third visible indicium indicative of a sterilized stage of the medical package in response to exposure to a temperature greater than or equal to a second temperature threshold.

This application claims the benefit of U.S. Provisional Application No. 62/396,654 entitled, “MEDICAL PACKAGE,” which was filed on Sep. 19, 2016 and is incorporated by reference herein in its entirety.

### TECHNICAL FIELD

This disclosure relates to a medical package.

### BACKGROUND

Medical packages may be used to house medical supplies in a sterile environment. In addition to helping to protect the medical supplies from damage that may be caused by handling or storage, medical packages may help preserve the sterility of medical supplies up to the time of use. For example, a medical package may house a medical device or other medical supply within a sealed space, and the medical package, including the medical device or other medical supply, may be sterilized prior to distribution. Thus, the medical package may maintain the sterility of medical supplies within a sealed environment prior to use of the medical device or other medical supply. At the time of use, a medical practitioner may retrieve the medical device or other medical supply from the medical package by opening the container in a sterile environment to help preserve the sterility of the medical supplies.

### SUMMARY

In some examples, this disclosure describes an example assembly that includes a medical package defining a housing configured to receive a medical supply. The medical package comprises a first sealable region and a second sealable region. The first and second sealable regions are configured to be sealed together to secure the medical supply within the housing. The medical package includes a thermal pattern configured to exhibit a first visible indicium indicative of an unsealed region between the first and second sealable regions. The thermal pattern is configured to irreversibly transform from the first visible indicium to a second visible indicium in response to exposure to a temperature greater than or equal to a first temperature threshold. The second visible indicium is indicative of a sealed region between the first and second sealable regions. The thermal pattern is also configured to irreversibly transform from the first visible indicium to a third visible indicium in response to exposure to a temperature greater than or equal to a second temperature threshold. The third visible indicium is indicative of a sterilized state of the medical package. At least a portion of the third visible indicium spaced from the first and second sealable regions, such that the third visible indicium may be separately viewed from the second visible indicium. In some examples, the first temperature threshold is greater than the second temperature threshold.

The example assemblies may be assembled, and the example articles according to the disclosure may be formed using any suitable technique. An example technique includes aligning a first sealable region of a medical package and a second sealable region of the medical package, and heating at least one of the first or second sealable regions to a sealing temperature to seal the first and second sealable regions together. In response to exposure to this sealing temperature, a thermal pattern visible on an outer surface of the medical package transforms from a first visible indicium to a second visible indicium indicative of the seal between the first and second sealable regions. The technique may also include sterilizing the medical supply within the package by exposing the medical package (including the medical supply therein) to a sterilization temperature, e.g., prior to or after sealing the package. In response to exposure to this sterilization temperature, the thermal pattern transforms from the first visible indicium to a third visible indicium indicative of a sterilized state of the medical package. The sterilization temperature may be less than the sealing temperature.

Clause 1: In some examples, an assembly comprises a medical package defining a housing configured to receive a medical supply. The medical package comprises a first sealable region and a second sealable region. The first and second sealable regions are configured to be sealed together to secure the medical supply within the housing. The medical package comprises a thermal pattern configured to exhibit a first visible indicium indicative of an unsealed region between the first and second sealable regions. The thermal pattern is configured to irreversibly transform from the first visible indicium to a second visible indicium indicative of a sealed region between the first and second sealable regions in response to exposure to a temperature greater than or equal to a first temperature threshold. The thermal pattern is configured to irreversibly transform from the first visible indicium to a third visible indicium indicative of a sterilized state of the medical package in response to exposure to a temperature greater than or equal to a second temperature threshold. At least a portion of the third visible indicium is spaced from the first and second sealable regions.

Clause 2: The assembly of clause 1, wherein the medical package comprises a first package portion comprising the first sealable region, and a second package portion comprising the second sealable region. The housing is defined between the first and second package portions.

Clause 3: The assembly of clauses 1 or 2, wherein the first and second sealable regions are along a periphery of the medical package.

Clause 4: The assembly of any of clauses 1 to 3, wherein the first and second sealable regions are configured to be sealed together to form a sterile barrier to the housing.

Clause 5: The assembly of any of clauses 1 to 4, wherein the first temperature threshold is less than or equal to a sealing temperature at which the first and second sealable regions seal together, and wherein the second temperature threshold is less than or equal to a sterilization temperature at which the medical supply is sterilized.

Clause 6: The assembly of clause 5, wherein the first temperature threshold is greater than the sterilization temperature.

Clause 7: The assembly of clause 6, wherein the sterilization temperature is about 49° C. (about 120° F.), wherein the first temperature threshold is about 66° C. (about 150° F.), and wherein the sealing temperature is about 138° C. (about 280° F.).

Clause 8: The assembly of any of clauses 1 to 7, wherein the thermal pattern comprises one or more of a thermally sensitive dye, a thermally sensitive pigment, or a thermally sensitive ink.

Clause 9: The assembly of any of clauses 1 to 8, wherein the thermal pattern comprises one or more of a substantially continuous line, a substantially continuous band, a logo, a word, or a phrase, a motif, or visual indicia, or a repeating pattern of the logo, the word, or the phrase.

Clause 10: The assembly of any of clauses 1 to 9, wherein a difference between the first, second, and third visible indicia is visually perceptible under one or both of visible light or ultraviolet light.

Clause 11: The assembly of any of clauses 1 to 10, wherein the medical package comprises one or more of a woven panel, a non-woven panel, a plastic panel, a polymer film, a paper film, a coated paper film, a metalized polymer film, a foil, a tray, a thermoform, a two-piece clamshell, or a folded clamshell.

Clause 12: The assembly of any of clauses 1 to 11, further comprising the medical supply enclosed within the housing.

Clause 13: The assembly of any of clauses 1 to 12, wherein the thermal pattern comprises a first thermal pattern defined by a first thermal composition and a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

Clause 14: An article comprising a first package portion comprising a first peripheral region, a second package portion comprising a second peripheral region substantially aligned with the first peripheral region, and a medical supply secured between the first and second package portions. At least one of the first or second peripheral regions comprises a thermal pattern configured to exhibit a first visible indicium indicative of an unsealed region between the first and second peripheral regions or an unsterilized state of the medical package. The thermal pattern is configured to irreversibly transform from the first visible indicium to a second visible indicium indicative of a sealed region between the first and second peripheral regions in response to exposure to a temperature greater than or equal to a first temperature threshold. The thermal pattern is configured to irreversibly transform from the first visible indicium to a third visible indicium indicative of a sterilized state of the medical package in response to exposure to a temperature greater than or equal to a second temperature threshold. At least a portion of the third visible indicium is spaced from the first and second sealable regions.

Clause 15: The article of clause 14, wherein the first temperature threshold is less than or equal to a sealing temperature at which the first and second peripheral regions seal together, and wherein the second temperature threshold is less than or equal to a sterilization temperature at which the medical supply is sterilized.

Clause 16: The article of clause 15, wherein the first temperature threshold is greater than the sterilization temperature.

Clause 17: The article of any of clauses 14 to 16, wherein the thermal pattern comprises one or more of a thermally sensitive dye, a thermally sensitive pigment, or a thermally sensitive ink.

Clause 18: The article of any of clauses 14 to 17, wherein the thermal pattern comprises one or more of a substantially continuous line, a substantially continuous band, a logo, a word, or a phrase, a repeating pattern of the logo, the word, or the phrase, a motif, or visual indicia.

Clause 19: The article of any of clauses 14 to 18, wherein a difference between the first, second, and third colors or patterns is visually perceptible under one or both of visible light or ultraviolet light.

Clause 20: The article of any of clauses 14 to 19, wherein one or both of the first and second package portion comprises one or more of a woven panel, a non-woven panel, a plastic panel, a polymer film, a paper film, a coated paper film, a metalized polymer film, a foil, a tray, a thermoform, a two-piece clamshell, or a folded clamshell.

Clause 21: The article of any of clauses 14 to 20, wherein the thermal pattern comprises a first thermal pattern defined by a first thermal composition and a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

Clause 22: A method comprising aligning a first sealable region of a medical package and a second sealable region of the medical package, wherein the medical package defines a housing configured to receive a medical supply; and heating at least one of the first or second sealable regions to a sealing temperature to seal the first and second sealable regions together. The medical package comprises a thermal pattern configured to exhibit a first visible indicium indicative of an unsealed region between the first and second sealable regions or an unsterilized state of the medical package. The thermal pattern is configured to irreversibly transform from the first visible indicium to a second visible indicium indicative of a sealed region between the first and second sealable regions in response to exposure to a temperature greater than or equal to a first temperature threshold. The thermal pattern is configured to irreversibly transform from the first visible indicium to a third visible indicium indicative of a sterilized state of the medical package in response to exposure to a temperature greater than or equal to a second temperature threshold. At least a portion of the third visible indicium is spaced from the first and second sealable regions.

Clause 23: The method of clause 22, wherein aligning the first sealable region of the medical package and the second sealable region of the medical package comprises aligning a first package portion with a second package portion, the housing being defined between the first and second package portions.

Clause 24: The method of clause 22 or clause 23, wherein heating the at least one of the first or second sealable regions to the sealing temperature seals the first and second sealable regions together to substantially close the housing.

Clause 25: The method of any of clauses 22 to 24, wherein the first and second sealable regions are configured to be sealed together to form a sterile barrier to the housing.

Clause 26: The method of any of clauses 22 to 25, wherein heating the at least one of the first and sealable regions to the sealing temperature comprises impulse sealing the first and second sealable regions.

Clause 27: The method of any of clauses 22 to 26, further comprising, before heating the at least one of the first and sealable regions to the sealing temperature, applying the thermal pattern to at least one of the first or second sealable regions.

Clause 28: The method of clause 27, wherein applying the thermal pattern comprises one or more of coating, stenciling, stamping, printing, or pad printing a thermal composition in a predetermined pattern.

Clause 29: The method of clause 27 or 28, wherein applying the thermal pattern comprises applying a first thermal pattern defined by a first thermal composition and applying a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

Clause 30: The method of any of clauses 22 to 29, wherein the first temperature threshold is less than or equal to the sealing temperature, wherein the second temperature threshold is less than or equal to a sterilization temperature at which the medical supply is sterilized, and wherein the first temperature threshold is greater than or equal to the sterilization temperature.

Clause 31: The method of any of clauses 22 to 30, further comprising: introducing a medical supply into the housing; and sterilizing the medical supply by at least exposing the medical supply and the medical package to a sterilization temperature for a predetermined duration of time, wherein the sterilization temperature is less than the first temperature threshold.

Clause 32: An assembly comprising: a medical package defining a housing configured to receive a medical supply, the medical package comprising a first sealable region and a second sealable region, wherein the first and second sealable regions are configured to be sealed together to secure the medical supply within the housing. The medical package comprises a first thermal pattern defined by a first thermal composition configured to exhibit a first visible indicium indicative of an unsealed region between the first and second sealable regions and irreversibly transform to a second visible indicium indicative of a sealed region between the first and second sealable regions in response to exposure to a temperature greater than or equal to a first temperature threshold. The medical package comprises a second thermal pattern defined by a second thermal composition configured to irreversibly transform to a third visible indicium indicative of a sterilized state of the medical package from a fourth visible indicium indicative of an unsterilized state of the medical package in response to exposure to a temperature greater than or equal to a second temperature threshold.

Clause 33: The assembly of clause 32, wherein at least a portion of the second thermal pattern is spaced from the first thermal pattern.

Clause 34: The assembly of clause 33, wherein the second thermal pattern is spaced in its entirety from the first thermal pattern.

Clause 35: The assembly of any of clauses claims 32 to 34, wherein at least a portion of the second thermal pattern is spaced from the first and second sealable regions.

Clause 36: The assembly of clause 35, wherein the second thermal pattern is spaced in its entirety from the first and second sealable regions.

Clause 37: The assembly of any of clauses 32 to 36, wherein the first temperature threshold is less than or equal to a sealing temperature at which the first and second sealable regions seal together, and wherein the second temperature threshold is less than or equal to a sterilization temperature at which the medical supply is sterilized.

Clause 38: The assembly of clause 37, wherein the first temperature threshold is greater than the sterilization temperature.

Clause 39: The assembly of clause 38, wherein the sterilization temperature is about 49° C. (about 120° F.), wherein the first temperature threshold is about 66° C. (about 150° F.), and wherein the sealing temperature is about 138° C. (about 280° F.).

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1A is a schematic and conceptual exploded perspective view of an example assembly that includes a medical package and a medical supply.

FIG. 1B is schematic and conceptual perspective view of the medical package of FIG. 1A in a sealed state.

FIG. 2A is a schematic and conceptual exploded perspective view of another example assembly that includes a medical package and a medical supply.

FIG. 2B is a schematic and conceptual perspective view of the medical package of FIG. 2A in a sealed state.

FIG. 3A is a schematic and conceptual perspective view of a first package portion of an example medical package, the first package portion including a substantially continuous thermal pattern.

FIG. 3B is a schematic and conceptual perspective view of the first package portion of FIG. 3A after exposure to sterilization and sealing temperatures.

FIGS. 4A, 4B, 4C, 4D, and 4E are schematic and conceptual top views of example thermal patterns indicating sealed, unsealed, sterilized, and unsterilized regions of a medical package.

FIG. 5 is a flow diagram illustrating an example method of manufacture of an example medical package.

FIG. 6 is a photograph illustrating an example panel including an example thermal pattern.

### DETAILED DESCRIPTION

A medical package defines a housing configured to receive and house one or more medical devices or other medical supplies. The medical package may be used to maintain the one or more medical supplies in a sterile environment, e.g., up to the point of use of the one or more medical supplies. The medical package comprises a first sealable region and a second sealable region. The first and second sealable regions are configured to be sealed together to secure the one or more medical supplies within the housing.

The medical package includes a thermal pattern, which is visible on an outer surface of the medical package. The thermal pattern may display an initial indicium, and transform or change to another indicium in response to exposure to a predetermined temperature. For example, the medical package may be exposed to a sterilization temperature at which a medical supply may be sterilized. The thermal pattern may transform or change to exhibit an indicium indicative of sterilization in response to exposure to the sterilization temperature. Thus, sterilization of the medical supply may be confirmed by verifying that the thermal pattern exhibits the indicium indicative of sterilization.

The medical package may be exposed to temperatures higher than sterilization temperatures, for example, higher temperatures that may be used to seal portions of the medical package. Further, in some cases, inadvertent exposure to high temperatures may affect the integrity of the medical package. To distinguish between exposures to different temperatures, the thermal pattern may transform to different indicia in response to different temperatures. For example, a sterilization temperature may be about 49° C. (about 120° F.), and a sealing temperature may be about 138° C. (about 280° F.). The thermal pattern may transform from the initial indicium (referred to herein as a “first visible indicium”) to a different indicium (referred to herein as a “third visible indicium”) in response to exposure to the sterilization temperature, and may further transform to another indicium (referred to herein as a “second visible indicium”) in response to exposure to the sealing temperature (or a temperature sufficiently higher than the sterilization temperature indicative of sealing).

While substantially all portions of the medical package may be exposed to a sterilization temperature during sterilization of the medical supply, only some portions of the medical package may be exposed to a sealing temperature during sealing of the medical package to a closed state. For example, only peripheral edge regions of the medical package that are sealed together may be exposed to the sealing temperature. Such selective exposure of portions of the package to the higher sealing temperatures may help protect the medical supply from exposure to the higher sealing temperature.

To visually distinguish different portions of the medical package exposed to different temperatures, regions of the thermal pattern may be spaced apart or away on a surface defined by the medical package. For example, one region of the thermal pattern may be disposed on sealable portions of the medical package, and another region of the thermal pattern may be spaced away from the sealable portions. In this way, only the region of the thermal pattern on or adjacent to the sealable portions may be exposed to the higher sealing temperatures, and the region of the thermal pattern spaced away from the sealable portions may only be exposed to sterilization temperatures.

Proper sealing and sterilization may be confirmed by verifying the presence of an indicium indicative of sealing only on the sealable portions, and verifying the presence of an indicium indicative of sterilization at other regions of the medical package. If other regions of the medical package are inadvertently exposed to a sealing temperature, they may display the indicium indicative of sealing, instead of the indicium indicative of sterilization. Such exposure may affect the integrity of the medical package or the medical supply, and medical packages displaying incorrect or unexpected indicia in different regions after processing may be discarded.

In some examples, a single, uniform thermal pattern may be applied to different surface regions of the medical package. The single thermal pattern may include at least one region spaced from the sealable portions of the medical package. The thermal pattern may be configured to transform from a first visible indicium to a second visible indicium in response to exposure to a temperature greater than or equal to a first temperature threshold. The second visible indicium is indicative of a sealed region between the first and second sealable regions. The thermal pattern may also be configured to irreversibly transform from the first visible indicium to a third visible indicium in response to exposure to a temperature greater than or equal to a second temperature threshold. The third visible indicium is indicative of a sterilized state of the medical package. The first and second temperature thresholds are respective temperature values, and may be predetermined prior to application of the thermal pattern on a medical package. Applying a single pattern may simplify manufacturing, for example, by only requiring a single printing or stamping step to apply the thermal pattern.

However, in other examples, different thermal patterns may be applied to different regions of the medical package. Applying different thermal patterns may help provide a better visible distinction or contrast between different regions of the medical package, and, as a result, the different temperature conditions the regions may have been exposed to in the course of processing.

The transformation or change of the thermal pattern to the different indicia may be successively irreversible. For example, once a region of the thermal pattern transforms to one indicium in response to exposure to a temperature greater than or equal to a temperature threshold, the thermal pattern may not revert back to an initial pattern or a previous pattern after the package cools down to an ambient temperature. However, the thermal pattern may exhibit further changes from one indicium to another indicium (different than the previous indicium) in response to exposure to successively higher temperatures. Thus, the presence of different visible indicia in different regions of the medical package may visually indicate different temperature exposures.

Monitoring the sterility of the medical supply enclosed within the housing of the package, e.g., on a manufacturing line, may be relatively difficult because it may require added inspection or biological sampling and analysis to determine the achievement of sterilization temperatures. Operators may be forced to open sealed packages to perform such inspection and analysis, which may lead to wastage of packages and entail additional inspection stages that may increase the manufacturing time.

An inadvertent failure to properly seal the medical package, for example, during manufacturing, may expose the enclosed medical supply to nonsterile conditions. Seal failure may result from improper sealing between first and second sealable regions of the medical package (e.g., a respective first peripheral edge region of a first package portion and a second peripheral edge region of a second package portion).

For example, a medical package may include a pouch. In some examples, the pouch may be formed from one or more polymeric sheets, for example, a spunbonded olefin sheet product, such as Tyvek® (DuPont USA, Wilmington, Del.), or sheets of other polymers, for example, polyethylene, or nylon. For example, the pouch may include a first sheet formed from Tyvek® and a second sheet formed from one of polyethylene or nylon. After a medical supply is placed into a housing of the pouch through an unsealed mouth of the pouch having three pre-sealed edges, the pouch may be sealed by sealing first and second peripheral edge regions of the pouch. The first and second peripheral edge regions may be sealed by thermal sealing. For example, impulse sealing may be used to expose at least one of the first or second peripheral edge regions to a predetermined sealing temperature that causes the first and second peripheral edge regions to seal, for example, by fusing or melting. However, some regions of the first and second peripheral edge regions may not be exposed to the sealing temperature during the impulse sealing. For example, debris, dust, or surface irregularities at the first or second peripheral edge regions or in a heating knife of an impulse sealer may prevent some regions of the first and second peripheral edge regions of the pouch intended to be sealed from attaining the sealing temperature during the impulse sealing. This may lead to the formation of unsealed regions, for example, unsealed islands, pockets, zones, or regions between the first and second peripheral edge regions. Contaminants may cross these one or more unsealed regions, which may compromise the sterility of the medical supply enclosed within the housing of the package.

Monitoring proper sealing of medical packages, e.g., on a manufacturing line, may be relatively difficult because it may require added inspection to determine whether all sealable edges were completely sealed. For example, operators may be forced to rely on unreliable or inefficient measures such as shaking or rattling medical packages, or a close visual inspection to verify complete sealing.

Further, verification of one of sealing or sterilization may not be indicative of the other of proper sealing of the other of sealing or sterilization. For example, even if sealing regions of the package are properly sealed, the contents of the medical package may not have been properly sterilized. Similarly, even if the contents of the medical package may be sterilized, the package may have been ineffectively or incompletely sealed, which could lead to a subsequent loss of sterility.

The thermal patterns described herein with respect to the example assemblies, articles, and techniques may allow for relatively easy, efficient, and reliable inspection of one of sealing or sterilization, or both of sealing and sterilization of medical packages, including medical pouches, medical trays, or other types of medical packages.

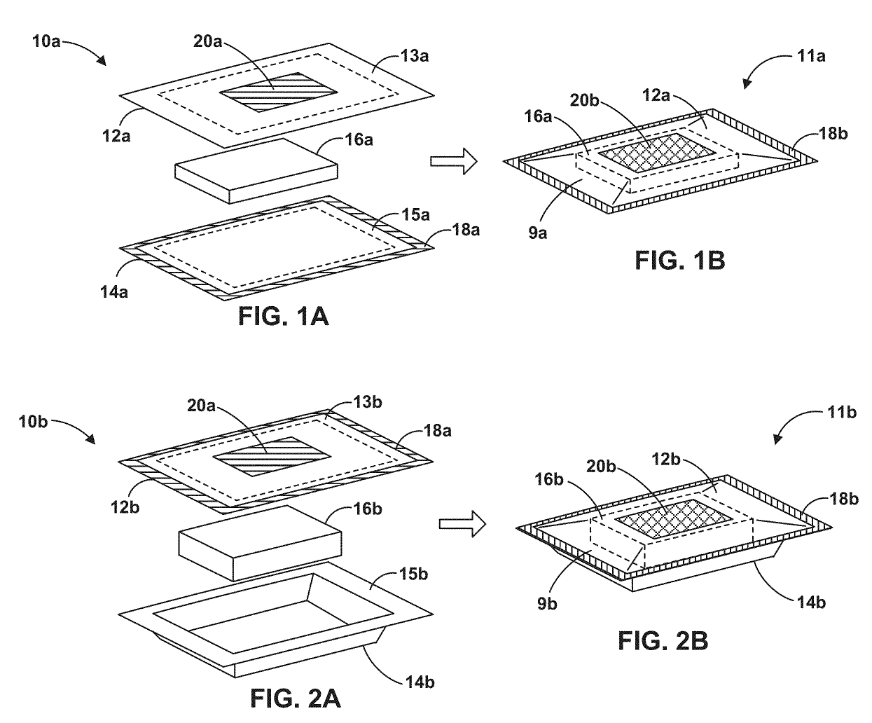

FIG. 1A is a schematic and conceptual exploded perspective view of an example assembly 10a. Assembly 10a includes a medical package including a first package portion 12a and a second package portion 14a. First package portion 12a includes a first sealable region 13a. Second package portion 14a including a second sealable region 15a. In some examples, first and second sealable regions 13a, 15a are on a periphery of the respective package portions 12a, 14a, and may, therefore, be referred to as peripheral regions. In other examples, first and second sealable regions 13a, 15a may be located in other locations of the medical package, such as away from the periphery of the package.

A thermal pattern 18a may be disposed on or applied to regions of one or both of first or second package portions 12a or 14a. In some examples, thermal pattern 18a may include a third region 20a spaced from first and second sealable regions 13a and 15a or from sealed region 18b (FIG. 1B). On exposure to different predetermined temperatures, for example, sealing or sterilization temperatures, different regions of thermal pattern 18a may transform to different visible indicia. For example, third region 20a may transform to an indicium indicative of sterilization, while regions of thermal pattern 18a on or adjacent first and second sealable regions 13a and 15a may transform to an indicium indicative of sealing.

Assembly 10a further includes a medical supply 16a disposed within housing 9a defined by the medical package (e.g., between package portions 12a, 14a). In some examples, one or both of first and second package portions 12a and 14a may include one or more of a woven panel, a non-woven panel, a plastic panel, a polymer film, a paper film, a coated paper film, a metalized polymer film, a foil, a tray, a thermoform, a two-piece clamshell, or a folded clamshell. For example, as shown in FIG. 1A, first package portion 12a may include a first panel of a pouch, and second package portion 14a may include a second panel of a pouch. However, in other examples, package portions 12a, 14a may have other configurations, as described in further detail with respect to FIGS. 2A and 2B.

First and second package portions 12a, 14a may be flexible in some examples, such that a medical package formed using the first and second package portions 12a, 14a is relatively flexible and conformable to a medical supply housed by the first and second package portions 12a, 14a. In other examples, however, first and second package portions 12a, 14a are relatively rigid (e.g., self-supporting), such that a medical package formed using the first and second package portions 12a, 14a may better protect a medical supply housed by the first and second package portions 12a, 14a. Further, in these examples, the first and second package portions 12a, 14a may provide structural stability and support to a medical supply by the first and second package portions 12a, 14a. In yet other examples, one of the first or second package portions 12a, 14a may be more flexible than the other, or more rigid than the other.

In some examples, first and second package portions 12a, 14a may have a substantially symmetric geometrical configuration relative to each other, as shown in FIG. 1A. While first and second package portions 12a, 14a are shown as discrete or separate in example assembly 10a of FIG. 1A, in some examples, first and second package portions 12a, 14a may be different portions of a continuous, unitary, or integrated medical package. For example, first and second package portions 12a, 14a may be foldable regions of a continuous sheet, web, or panel. Second sealable region 15a is configured to substantially align with first sealable region 13a. For example, first and second sealable regions 13a, 15a may have geometrically similar, superimposable, or otherwise alignable respective surfaces. In some examples, first and second sealable regions 13a, 15a may substantially extend about respective peripheries of the first and second package portions 12a, 14a, as shown in FIG. 1A. However, in other examples, one or both of the first and second sealable regions 13a, 15a may extend only across a predetermined respective portion of first and second package portions 12a, 14a. For example, one or both of the first and second sealable regions 13a, 15a may extend only across respective single edge regions of first and second package portions 12a, 14a, or only two or three edge regions of and second package portions 12a, 14a.

First and second sealable regions 13a, 15a are configured to be sealed together to help secure medical supply 16a within housing 9a defined between first and second package portions 12a, 14a, as shown in FIG. 1B. FIG. 1B is schematic and conceptual perspective view of a sealed sterile medical article 11a, that includes first package portion 12a and second package portion 14a (not visible in FIG. 1B) enclosing medical supply 16a in a sterile environment.

In some examples, one or both of first and second sealable regions 13a, 15a may be formed from a material that is configured to melt, soften, fuse, or otherwise be susceptible to sealing to the other of first and second sealable regions 13a, 15a in response to being exposed to or heated to a predetermined sealing temperature. For example, an impulse sealer may cause a heated knife edge to press against one or both of first and second sealable regions 13a, 15a, raising the temperature of the material of the regions 13a, 15a to at least the sealing temperature. Portions of at least one of first and second sealable regions 13a, 15a that are exposed to or heated to the sealing temperature may form a seal with a respective region of the other of first and second sealable regions 13a, 15a. However, portions of sealable regions 13a, 15a that fail to attain the sealing temperature may fail to seal, leading to the formation of unsealed regions of the medical package.

At least one of first and second sealable regions 13a, 15a includes thermal pattern 18a. In some examples, thermal pattern 18a includes one or more of a thermally sensitive dye, a thermally sensitive pigment, or a thermally sensitive ink. In some examples, thermal pattern 18a may be formed from one or more thermal compositions. For example, a thermal composition may include heat sensitive permanent color change ink obtained from LCR Hallcrest (Glenview, Ill.). In some examples, pattern 18a may be defined by a single thermal composition. The single thermal composition may be configured to exhibit at least one color transformation in response to exposure to a predetermined temperature.

In other examples, the single thermal composition may be configured to exhibit more than one color transformations, each series or pairs of color transformations being in response to exposure to temperatures greater than or equal to different predetermined temperature thresholds. For example, the single thermal composition may exhibit a first initial color, a second color in response to exposure to a temperature greater than or equal to a first temperature threshold, a third color in response to exposure to a temperature greater than or equal to a second temperature threshold, and the like. Such a single thermal composition may include, for example, a combination of thermal inks, pigments, or dyes, responsive to the different temperatures, or a single ink, pigment, or dye capable of exhibiting different chemical structures or other configurations responsive to different temperatures.

In other examples, different regions of pattern 18a may be defined by different thermal compositions. For example, a first region of pattern 18a may be defined by a first thermal composition configured to transform to a different color in response to exposure to a temperature greater than or equal to a first temperature threshold, a second region of pattern 18a may be defined by a second thermal composition configured to transform to a different color in response to exposure to a temperature greater than or equal to a second temperature threshold, and so on, where the first temperature threshold is greater than the second temperature threshold. In these examples, the first region of pattern 18a does not include or does not significantly include a significant amount of the second thermal composition (e.g., to result in a visible change of color corresponding to the second thermal composition), and the second region of pattern 18a does not include or does not significantly include a significant amount of the first thermal composition.

In some examples, thermal pattern 18a may be applied to a surface defined by first or second package portions 12a, 14a, for example, one or both of first and second sealable regions 13a, 15a. In the example shown in FIG. 1A, in an unsealed configuration, first sealable region 13a does not exhibit any region of thermal pattern 18a, while second sealable region 15a exhibits a region of thermal pattern 18a, and third region 20a exhibits another region of thermal pattern 18a. In other examples, in an unsealed configuration, second sealable region 15a may not exhibit any region of thermal pattern 18a, while first sealable region 13a exhibits a region of thermal pattern 18a, and third region 20a exhibits another region of thermal pattern 18a.

Thermal pattern 18a may be applied by printing, stamping, or any other suitable technique. In some examples, thermal pattern 18a may be applied on a substrate, for example, one or more strips or labels, that is applied to the surface of a medical package. In some examples, one or more regions of thermal pattern 18a may be applied to a surface spaced from one or both of first and second sealable regions 13a, 15a.

Thermal pattern 18a may be configured to indicate the exposure of portions of medical package 10a to different temperatures. For example, one or more regions of thermal pattern 18a may exhibit the first visible indicium, indicating no exposure to elevated temperatures. One or more of these regions of thermal pattern 18a may transform to a different visible indicium in response to exposure of those regions to a particular temperature. For example, a region of thermal pattern 18a may transform to the second visible indicium in response to exposure to a temperature greater than or equal to a first temperature threshold, a different region of thermal pattern 18a may transform to the third visible indicium in response to exposure to a temperature greater than or equal to a second temperature threshold, which may be less than the first temperature threshold. Thus, the state of transformation of different regions of thermal pattern 18a may be indicative of exposure of the different regions to different temperatures. In this way, thermal pattern 18 may indicate different conditions or states of the medical package associated with the respective temperature thresholds. For example, the first temperature threshold may be indicative of sealing, and the second temperature threshold may be indicative of sterilization.

In some examples, the transformation of one or more regions of thermal pattern 18a may be indicative of states of the medical package or the medical supply other than sealing or sterilization. For example, exposing a region of medical package to a temperature higher than predetermined softening, melting, or charring temperatures may result in a change in integrity of the medical package or the medical supply. The change in integrity may adversely affect the usability of the medical supply. In some examples, a third temperature threshold may be indicative of softening point (for example, a temperature above a glass transition temperature of a polymeric component of medical package or the medical supply), a fourth temperature threshold may be indicating of a melting point of a component of the medical package or medical supply, and a fifth temperature threshold may be indicative of the onset of charring, burning, undesirable oxidation, or other loss of integrity of the medical package. In some such examples, thermal pattern 18a may be configured to transform to one or more of a fourth, fifth, or sixth visible indicium respectively indicative of softening, melting, or loss of integrity of the medical package or the medical supply in response to exposure to a temperature greater than or equal to the respective temperature thresholds.

In some examples, the sealing temperature may be higher than a temperature threshold to which the medical supply within the medical package may be exposed in order to maintain the integrity (e.g., operability) of the medical supply housed within the medical package. For example, only the first and second sealing regions 13a and 15a may be exposed to sealing temperatures, while the rest of the medical package may be exposed only to temperatures lower than the sealing temperature by a predetermined difference. In some such examples, transformation of thermal pattern 18a to the second visible indicium (indicative of sealing) in regions spaced from first and second sealing regions 13a and 15a may be indicative of an undesirable change in integrity of the medical supply or the package in regions spaced from first and second sealing regions 13a and 15a. Thus, thermal pattern 18a may be configured to indicate different states of the medical package, or to indicate exposure of different regions or portions the medical package to different temperatures.

The thermal patterns described herein for indicating exposure of a medical package to a sealing temperature and a sterilization temperature may be used with any suitable medical package configuration. Thus, in alternate examples, assembly 10a of FIG. 1A may assume other suitable geometric forms to result in predetermined medical package geometries, including pouches, trays, or other packages having any predetermined geometries.

For example, FIG. 2A is a schematic and conceptual exploded perspective view of an example assembly 10b for forming a medical package including a tray defining a housing configured to receive and house a medical supply. FIG. 2B is a schematic and conceptual perspective view of sealed a sterile medical article 11b. Assembly 10b includes a first package portion 12b, for example, shaped as a lid, and a second package portion 14b, for example, shaped as a tray, as shown in FIG. 2A. Once assembled, assembly 10b may define an article 11b as shown in FIG. 2B. First package portion 12b includes a first sealable region 13b, and second package portion 14b includes a second sealable region 15b. Together, first package portion 12b and second package portion 14b define housing 9b configured to receive and house medical supply 16b.

Thermal pattern 18a may be disposed on or applied to regions of one or both of first or second package portions 12b or 14b. In some examples, thermal pattern 18a may include a third region 20a spaced from first and second sealable regions 13b and 15b or from sealed region 18b (FIG. 2B). On exposure to different predetermined temperatures, for example, sealing or sterilization temperatures, different regions of thermal pattern 18a may transform to different visible indicia. For example, third region 20a may transform to an indicium indicative of sterilization, while regions of thermal pattern 18a on or adjacent first and second sealable regions 13b and 15b may transform to an indicium indicative of sealing.

While thermal patterns 18a shown in FIGS. 1A-2B may include spaced apart regions, in other examples, the thermal pattern may be substantially continuous across a surface defined by an article. As with thermal pattern 18a, the substantially continuous thermal pattern may be configured to transform from a first indicium (or a third indicium) to a second indicium in response to being exposed to a temperature greater than or equal to a first temperature threshold, and transform from the first indicium to a second indicium in response to being exposed to a temperature greater than or equal to a second temperature threshold less than the first temperature threshold.

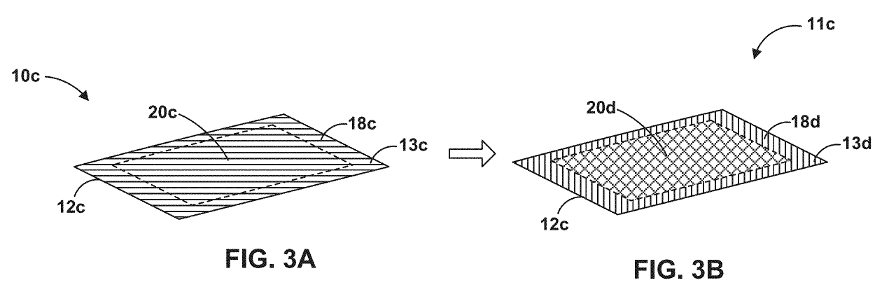

FIG. 3A is a schematic and conceptual perspective view of an example first package portion 12c of an example assembly 10c. Example assembly 10c is substantially similar to example assembly 10a of FIG. 1A, except that thermal pattern 18c in example assembly 10c is substantially continuous across first package portion 12c. FIG. 3B is a schematic and conceptual perspective view of first package portion 12c of FIG. 3A after exposure to sterilization and sealing temperatures in a sealed article 11c. As seen in FIG. 3B, due to the manner in which the medical package is sealed, a third region 20c (FIG. 3A) of thermal pattern 18c is exposed only to a sterilization temperature and not a sealing temperature. As a result, third region 20c changes to region 20d exhibiting the third visible indicium indicative of sterilization, while portions of thermal pattern 18c adjacent sealing region 13c are exposed to sealing temperatures and changes to the second visible indicium 18d indicative of sealing.

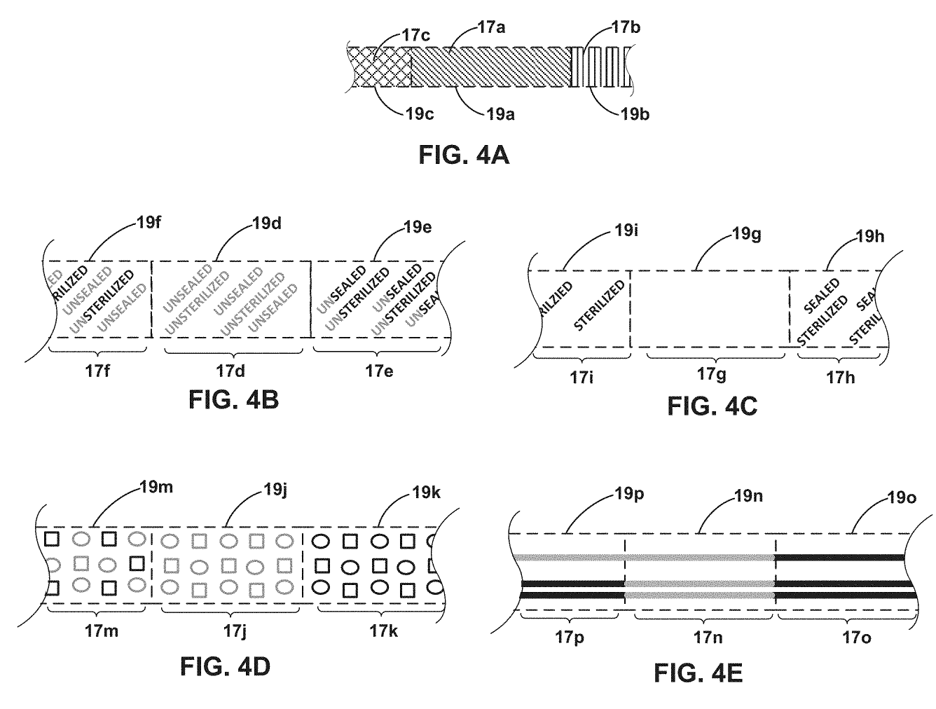

The visible indicia provided by thermal pattern 18a may take the form of any suitable visible indicia. FIGS. 4A, 4B, 4C, 4D, and 4E are schematic and conceptual top views of example visible indicial provided by thermal patterns. The thermal patterns shown in FIGS. 4A-4E indicate sealed and unsealed regions and sterilized and unsterilized states of the medical package. While FIGS. 4A-4E are described with reference to thermal patterns 18a and 18b of articles 10a and 11a of FIGS. 1A and 1B, the descriptions apply to thermal patterns 18a and 18b of articles 10b and 11b of FIGS. 2A and 2B, and to thermal patterns 18c and 18d of articles 10c and 11c of FIGS. 3A and 3B.

Thermal pattern 18a is configured to exhibit a first visible indicium, for example, first visible indicium 17a shown in FIG. 4A, indicative of an unsealed region 19a or an unsterilized state of the medical package. The first visible indicium (or other visible indicia according to the disclosure) may include any suitable color and/or pattern that can be distinguished from a standard indicium, a comparative indicium, an adjacent indicium, a surrounding or adjacent region of the medical package, or any other indicia, by visual inspection. The unsealed region 19a is defined by portions of first and second sealable regions 13a and 15a that are not sealed together. Thermal pattern 18a is configured to transform from the first visible indicium to a second visible indicium, for example, second visible indicium 17b shown in FIG. 4A, in response to exposure to a temperature greater than or equal to a first temperature threshold. Second visible indicium 17b is indicative of a sealed region of the medical package, for example a sealed region 19b (shown in FIG. 4A) defined by portions of first and second sealable regions 13a and 15a sealed together.

Thermal pattern 18a may be configured to transform from the first visible indicium to a third visible indicium, for example, third visible indicium 17c (shown in FIG. 4A) in response to exposure to a temperature greater than or equal to a second temperature threshold. Third visible indicium 17c is indicative of a sterilized state of the medical package. In some examples, at least a portion of third visible indicium 17c may be disposed in a region 19c or a portion of thermal pattern 18a spaced from first and second sealable regions 13a and 15a or from sealed region 19b. For example, as shown in FIG. 1A, thermal pattern 18a may include third region 20a spaced from first and second sealable regions 13a and 15a or from sealed region 19b. In some examples, a periphery or a boundary defined by third portion 20a may be at least about 1.25 centimeters (cm) (about 0.5 inches), or at least about 2.5 cm (about 1 inch), or at least about 5 cm (about 2 inches) away from a periphery or a boundary defined by first sealable region 13a or second sealable region 15a. In some examples, third region 20a of thermal pattern 18a may be adjacent to or surrounded by one or more portions of first and second sealable regions 13a and 15a or sealed region 19b. As seen in FIG. 1A, portions of thermal pattern 18a at or adjacent first and second sealable regions 13a and 15a and third region 20a of thermal pattern 18a spaced from first and second sealable regions 13a and 15a may exhibit first visible indicium 17a, before sealing and sterilization. In some examples, third region 20a of thermal pattern 18a may be spaced sufficiently away from first and second sealable regions 13a and 15a such that even when first and second sealable regions 13a and 15a are exposed to sealing temperatures or conditions, third region 20a is not exposed to the same sealing temperatures or conditions.

While thermal pattern 18a may include third region 20a, for example, being defined by the same thermal composition, in some examples, third region 20a may be a second thermal pattern defined by a different thermal composition than thermal pattern 18a. For example, thermal pattern 18a may be a first thermal pattern defined by a first thermal composition configured to exhibit the first visible indicium indicative of an unsealed region between first and second sealable regions 13a and 15a and the second visible indicium indicative of a sealed region between the first and second sealable regions. Third region 20a may be a second thermal pattern defined by a second thermal composition configured to exhibit a third visible indicium indicative of a sterilized state of the medical package and a fourth visible indicium indicative of an unsterilized state of the medical package. In some examples, the fourth visible indicium may be the same as the first visible indicium. For example, the first visible indicium indicative of an unsealed state may also be indicative of an unsterilized state.

In some examples, at least a portion of the second thermal pattern is spaced from the first thermal pattern. In some such examples, second thermal pattern may include third region 20a spaced in its entirety from first thermal pattern 18a, for example, as shown in FIG. 1A. In other examples, only a portion of the second thermal pattern is spaced from first thermal pattern 18a, and another portion of the second thermal pattern is directly adjacent to (e.g., sharing a border with) first thermal pattern 18a. In addition, in some examples, at least a portion of the second thermal pattern is spaced from first and second sealable regions 13a and 15a. In some examples, the second thermal pattern is spaced in its entirety from first and second sealable regions 13a and 15a, for example, as shown in FIG. 1A.

While respective first, second, and third visible indicium 17a, 17b, and 17c are shown as substantially uniform bands in the example article of FIG. 4A, in some examples, one or more of first, second, and third visible indicium 17a,17b, and 17c of thermal pattern 18a may include one or more of a substantially continuous line, a substantially continuous band, a logo, a word, or a phrase, a repeating pattern of the logo, the word, or the phrase, a motif, or visual indicia.

For example, as shown in FIG. 4B, in some examples, a first visible indicium 17d defined by a first portion 19d of thermal pattern 18a may include repeated word(s), for example, one or both of “unsealed” and “unsterilized,” while a second visible indicium 17e defined by a second portion 19e of thermal pattern 18a may include one or more repeated words, for example, “sealed,” and a third visible indicium 17f defined by a third portion 19f of thermal pattern 18a may include another repeated word, for example, “sterilized”. In some examples, second visible indicium 17e may include two repeated words, for example, both “sealed” and “sterilized.”

In the example shown in FIG. 4B, thermal pattern 18a is configured such that only the portion “sealed” of the word “unsealed” changes in intensity or saturation to change to the second visible indicium and only the portion “sterilized” changes in intensity or saturation to change to the third visible indicium in response to exposure of a region of thermal pattern 18a to a sealing temperature or another temperature greater than or equal to a first temperature threshold. For example, the “sealed” portion of the word “unsealed” or the “sterilized” portion of the word “unsterilized” may turn visibly turning darker or a different color to be relatively visually more prominent compared to the portion “un” of the word “unsealed” or “unsterilized” in response to being exposed to or heated to a predetermined temperature (for example, a sealing temperature or a sterilization temperature).

In other examples, for example, as shown in FIG. 4C, a first visible indicium 17g defined by a first portion 19g of thermal pattern 18a may include a uniform pattern with no visible words, while a second visible indicium 17h defined by a second portion 19h of thermal pattern 18a may include repeated words, for example, one or both of “sealed” or “sterilized.” In some examples, the first visible indicium may include suitable words such as “void,” “non-sterile,” “do not use,” “discard,” or other suitable verbal or graphic indicia may be used to indicate the presence of unsealed or unsterile regions. However, other colors or patterns may be used in other examples.

For example, as shown in FIG. 4D, the intensity or presence of a first plurality of indicia (for example, ellipsoidal indicia) may be indicative of sealing, while the intensity or presence of a second plurality of indicia (for example, rectangular indicia) may be indicative of sterilization. Thus, a first visible indicium 17j defined by a first portion 19j of thermal pattern 18a may include low intensity indicia or no visible indicia. Second visible indicium 17k defined by a second portion 19k of thermal pattern 18a may include visible or higher intensity (for example, one or both of higher saturation or higher darkness) of the first plurality of indicia, indicative of sealing at second portion 19k. Third visible indicium 17m defined by a third portion 19m of thermal pattern 18a may include visible or higher intensity (for example, one or both of higher saturation or higher darkness) of the second plurality of indicia, indicative of sterilization at third portion 19m.

In some examples, as shown in FIG. 4E, one or more indicia may include a substantially continuous line or band, which may wrap fully or partially around a periphery of a surface of medical package. The intensity or presence of a a first line or band (for example, a single line) may be indicative of sealing, while the intensity or presence of a second line or band (for example, a double line) may be indicative of sterilization. Thus, a first visible indicium 17n defined by a first portion 19n of thermal pattern 18a may include low intensity indicia or no visible indicia, and no lines may be visible. Second visible indicium 17o defined by a second portion 19o of thermal pattern 18a may include visible or higher intensity (for example, one or both of higher saturation or higher darkness) of the first line (or single line), indicative of sealing at second portion 19o. Third visible indicium 17p defined by a third portion 19p of thermal pattern 18a may include visible or higher intensity (for example, one or both of higher saturation or higher darkness) of the second line (for example, double line), indicative of sterilization at third portion 19p.

Disruptions or changes in intensity along the first line or single line may thus be indicative of unsealed portions along sealable regions 13a or 15a. Disruptions or changes in intensity along the second line or double line may thus be indicative of unsterilized regions. The second line or band may be adjacent to the first line or band, while being spaced from the first line or band, as shown in FIG. 4E. However, in other examples, the second line or band may be further spaced from the first line or band. For example, the second line or band may be disposed in an interior surface region, similar to a periphery defined by third region 20a.

While in some examples, as shown in FIGS. 4A-4E, the change in visible indicium from the first visible indicium to the second or third visible indicium may be visually perceptible under ambient or visible light, in other examples, the change in visible indicium may only be visible under ultraviolet light or another predetermined wavelength band of light. For example, an operator, medical practitioner, a consumer, or an optical sensor may scan medical packages after sealing under visible or ultraviolet light to verify that proper sealing of a medical package, for example, article 11a of FIG. 1B. In some examples, one or more of the first, second, or third visible indicia may not be visible under ambient light, and others of the first, second, or third visible indicia may be visible under ambient light. Thus, a transformation of thermal pattern 18a may manifest as a change of an invisible pattern to a visible pattern, or vice versa.

In some examples, thermal pattern 18a comprises a thermal composition configured to irreversibly (e.g., permanently) change color, for example, one or more of hue, saturation, or brightness of the color. The change in color transforms first visible indicium 17a to the second visible indicium 17b or third visible indicium 17c (or their variants according to the disclosure) in response to being exposed to a temperature greater than or equal to a temperature threshold. For example, the temperature threshold may be greater than or equal to a sealing temperature at which portions of the medical package seal together.

In some examples, the surface on which thermal pattern 18a is disposed may not achieve the actual sealing temperature, for example, because thermal pattern 18a may be applied on a surface that is opposite to the sealed surface. However, in such examples, the temperature of thermal pattern 18a may still sufficiently rise above room or ambient temperature to some temperature less than the sealing temperature, but still sufficiently higher than ambient or room temperature. Therefore, in some examples, the first temperature threshold may be less than or equal to the sealing temperature. In some examples, the first temperature threshold may be relatively close to the sealing temperature. For example, the difference between the temperature threshold and the sealing temperature may be less than about 50° C., 20° C., 10° C., 5° C., 1° C., or 0.5° C.

Portions of medical packages may be exposed to elevated temperatures during processing steps other than sealing. For example, one or both of first and second sealable regions 13a and 15a of the medical package shown in FIG. 1A may be exposed to elevated temperatures during sterilization of medical supply 16a. To help avoid false indications of sealing resulting from such elevated temperatures, in some examples, the first temperature threshold is greater than a predetermined sterilization temperature, which may be the minimum temperature at which the medical supply enclosed within housing 9a of the medical package is sterilized. In some examples, the first temperature threshold may be greater than the sterilization temperature by at least about 20° C., 10° C., 5° C., or 1° C. For example, the sterilization temperature may be about 49° C. (about 120° F.), the first threshold temperature may be about 66° C. (about 150° F.), and the sealing temperature may be about 138° C. (about 280° F.).

As seen in FIGS. 4A-4E, the presence of the first, second, or third visible indicium may be indicative of one or more of unsealed, sealed, sterilized, or unsterilized regions or conditions of the medical packages, for example, as described with reference to FIGS. 1A, 1B, 2A, 2B, 3A, and 3B. Because sealing temperatures may be higher than sterilization temperatures, regions exposed to sealing temperatures may also experience interim sterilization temperatures, and indicate both sealing and sterilization. In contrast, regions or portions of the medical package spaced or relative remote from sealed portions of the medical package may not experience sealing temperatures, and may thus be indicative of exposure of the medical package to sterilization temperatures lower than sealing temperatures.

In some examples, example assembly 10a of FIG. 1A including thermal pattern 18a may be used to produce example article 11 a of FIG. 1B, including thermal pattern 18b, as shown in FIG. 1B. A user may visually inspect thermal pattern 18b to relatively quickly confirm sealing of substantially an entire region of first and second sealable regions 13a and 15a of the medical package of assembly 10a to form respective first and second sealed regions of article 11a. In some examples, thermal pattern 18b may include a portion 20b spaced from first and second sealable regions 13a and 15a of article 11a. As seen in FIG. 1B, portion 20b of thermal pattern 18b may exhibit the third visible indicium indicative of a sterilized state of the medical package, while portions of thermal pattern 18b at or adjacent the first and second sealed regions of article 11 a may exhibit the second visible indicium indicative of sealing.

In some examples, assembly 10a may include more than one thermal pattern. For example, assembly 10 may include a plurality of thermal patterns, each pattern of the plurality of thermal pattern associated with a respective temperature threshold of a plurality of temperature thresholds. As an example, a first thermal pattern may change visible indicium at a temperature greater than or equal to a first temperature threshold, and a second thermal pattern may change visible indicium at a temperature greater than or equal to a second temperature threshold. The temperature thresholds may be associated with any selected thermal phenomenon or a selected stage of manufacturing. For example, the first temperature threshold may be less than or equal to the sealing threshold, but greater than the sterilization temperature, while the second temperature threshold may be less than or equal to the sterilization temperature. For example, the first temperature threshold may be within a predetermined range of the sterilization temperature. For example, the first temperature threshold may be at least 0.5° C., 1° C., 5° C., 10° C., 20° C., or 50° C., greater than the sterilization temperature. Thus, in some examples, example assemblies and articles may indicate proper sterilization and sealing.

Thus, example assemblies and articles according to the disclosure may be used to verify proper sealing and sterilization of medical packages. Example techniques described below may be used to assemble example assemblies, or form example articles according to the disclosure.

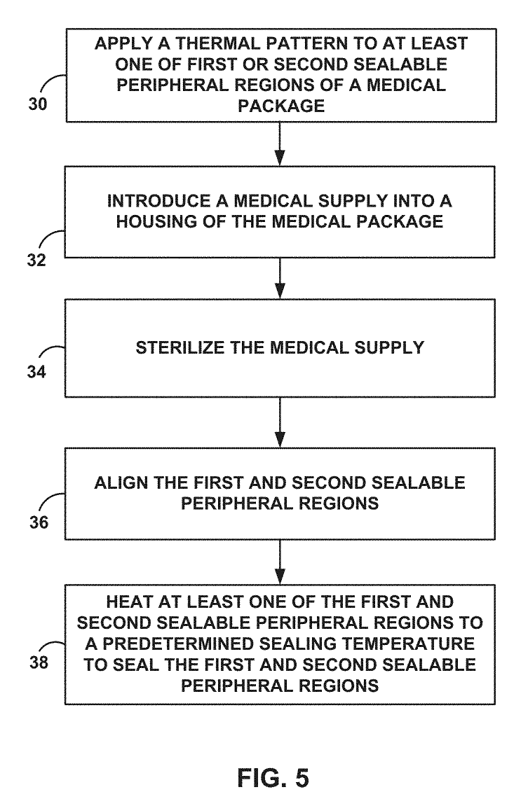

FIG. 5 is a flow diagram illustrating an example technique of manufacture of an example medical package. The example technique of FIG. 5 is described with reference to example assembly 10a of FIG. 1A and example article 11a of FIG. 1B. However, the example technique of FIG. 5 may be used to assemble any example assemblies or to form any example articles according to the disclosure.

The example technique of FIG. 5 includes applying thermal pattern 18a to at least one of first or second sealable regions 13a and 15a of a medical package (30). In some examples, the applying (30) may further include applying at least a third region 20a of thermal pattern 18a spaced from first and second sealable regions 13a and 15a. In some examples, applying thermal pattern 18 may include one or more of coating, stenciling, stamping, printing, or pad printing a thermal composition in a predetermined pattern. In some examples, the thermal composition is configured to change color, for example, one or more of hue, saturation, or brightness, transforming the first visible indicium to the second visible indicium, in response to being exposed to a temperature greater than or equal to a first temperature threshold (e.g., a temperature greater than or equal to a sterilization temperature, but less than or equal to a sealing temperature). The thermal composition is configured to change color to a different color, transforming the first visible indicium to the third visible indicium, in response to being exposed to a temperature greater than or equal to a second temperature threshold (e.g., a temperature greater or equal to the sterilization temperature). The thermal composition may include a temperature-dependent color changing component, for example, one or more of a thermally sensitive dye, a thermally sensitive pigment, or a thermally sensitive ink. In some examples, the thermal composition may include additional components, for example, one or more of solvents, suspensions, non-thermal inks, dyes, or pigments, viscosifiers, stabilizers, or other additives.

In some examples, the example technique of FIG. 5 may include introducing medical supply 16a into housing 9a of the medical package (32). For example, an operator, robot, or tool may one or more of align, position, or orient medical supply 16a relative to first and second package portions 12a and 14a, and bring first and second package portions 12a and 14a sufficiently close to at least temporarily retain medical supply 16a between first and second package portions 12a and 14a.

In some examples, the example technique of FIG. 5 may include sterilizing medical supply 16a (34). For example, medical supply 16a may be exposed to a predetermined sterilization temperature for a predetermined period of time. For example, the sterilization temperature may be about 49° C. (about 120° F.). In some examples, the period of time may be about 1 second, about 10 seconds, or about 1 minute or more. In some examples, in addition to, or instead of, exposing medical supply 16a to the sterilization temperature, the example technique of FIG. 5 may include one or more of microwave, steam, or ozone sterilization, or other sterilization techniques. The sterilizing may also include sterilizing the medical package, for example, first and second package portions 12a and 14a, to provide a sterile environment within housing 9a.

In some examples, the sterilizing may be performed after sealing medical supply 16a in the medical package. In other examples, the sterilizing may be performed before sealing medical supply 16a in the medical package. In some examples, the thermal composition is configured to change color, transforming the first visible indicium to the third visible indicium in response to being exposed to a temperature greater than or equal to a second temperature threshold, which may be, for example, the sterilization temperature or within a predetermined temperature range of the sterilization temperature. In some examples, on further heating to a sealing temperature, the thermal composition may change color to a different color, transforming the third visible indicium to the second visible indicium in response to being exposed to a temperature greater than or equal to the first temperature threshold, which is greater than the second temperature threshold.

While applying (30) may include applying a single thermal pattern, in some examples, applying (30) may include applying more than one thermal patterns. For example, applying the thermal pattern (30) may include applying a first thermal pattern defined by a first thermal composition and applying a second thermal pattern defined by a second thermal composition. The first thermal pattern may be configured to transform to the second visible indicium in response to exposure to a temperature greater than or equal to the first temperature threshold, and the second thermal composition may be configured to transform to the third visible indicium in response to exposure to a temperature greater than or equal to a second temperature threshold, which can be less than the first temperature threshold.

In some examples, the example technique of FIG. 5 includes aligning first sealable region 13a of first package portion 12a and second sealable region 15a of second package portion 14a (36). For example, an operator, robot, or tool may one or more of align, position, or orient first and second sealable regions 13a and 15a so that they may be sealed. At least one of first and second sealable peripheral regions 13a and 15a can be heated to the sealing temperature to seal first and second sealable regions 13a and 15a together (38). In some examples, the heating may include one or more of impulse sealing, laser sealing, ultrasound sealing, or other sealing techniques. For example, a thermal knife edge heated to the sealing temperature, for example, about 138° C. (about 280° F.), may be pressed against first and second sealable regions 13a and 15a to cause one or both of first and second sealable regions 13a and 15a to heat to the sealing temperature. Regions of at least one of first and second sealable regions 13a and 15a that attain the sealing temperature may soften, melt, fuse, or otherwise turn susceptible to seal against the other of first and second sealable regions 13a and 15a to form article 10b.

While in the example technique of FIG. 5, the heating (38) may be conducted after the sterilizing (34), in some examples, the heating (38) may be conducted before the sterilizing (34).

In some examples, the example technique of FIG. 5 may include, after the sealing, inspecting thermal pattern 18b of article 10b. For example, an operator or sensor may inspect thermal pattern 18b under one or both of visible or ultraviolet light to determine whether substantially all regions of thermal pattern 18b along or adjacent one or more sealing regions exhibit the second visible indicium, indicating a proper seal, or whether any region of thermal pattern 18b exhibits the first visible indicium, indicating an unsealed region. In some examples, an operator or sensor may inspect thermal pattern 18b under one or both of visible or ultraviolet light to determine whether substantially a portion or region of thermal pattern 18b spaced from one or more sealing regions exhibits the third visible indicium, indicating a proper sterilization state of the medical package, or whether any region of thermal pattern 18b exhibits the first color, indicating an unsterilized state of the medical package. Based on the result of the inspection, article 10b may be discarded, or accepted for storage, further processing, or use.

Thus, example techniques according to the disclosure may be used to assemble example articles that include thermal patterns indicative of seal integrity.

### EXAMPLES

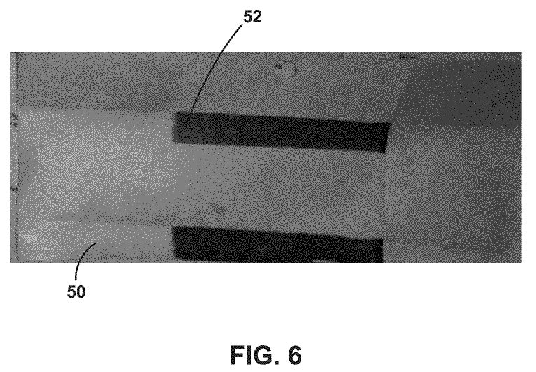

FIG. 6 is a photograph illustrating an example panel 50 including an example thermal pattern 52. Thermal pattern 52 exhibits a uniform color change on exposure to a temperature greater than a first threshold temperature, indicating that the sealing temperature was achieved across a sealing region of panel 50.

Various examples have been described. These and other examples are within the scope of the following claims.

What is claimed is:

1. An assembly comprising:
a medical package defining a housing configured to receive a medical supply, the medical package comprising a first sealable region and a second sealable region, wherein the first and second sealable regions are configured to be sealed together to secure the medical supply within the housing,
wherein the medical package comprises a thermal pattern configured to:
exhibit a first visible indicium indicative of an unsealed region between the first and second sealable regions or an unsterilized state of the medical package,
irreversibly transform from the first visible indicium to a second visible indicium indicative of a sealed region between the first and second sealable regions in response to exposure to a temperature greater than or equal to a first temperature threshold, and irreversibly transform from the first visible indicium to a third visible indicium indicative of a sterilized state of the medical package in response to exposure to a temperature greater than or equal to a second temperature threshold, wherein at least a portion of the third visible indicium is spaced from the first and second sealable regions.

2. The assembly of claim 1, wherein the medical package comprises:
a first package portion comprising the first sealable region; and
a second package portion comprising the second sealable region, wherein the housing is defined between the first and second package portions.

3. The assembly of claim 1, wherein the first and second sealable regions are along a periphery of the medical package.

4. The assembly of claim 1, wherein the first and second sealable regions are configured to be sealed together to form a sterile barrier to the housing.

5. The assembly of claim 1, wherein the first temperature threshold is less than or equal to a sealing temperature at which the first and second sealable regions seal together, and wherein the second temperature threshold is less than or equal to a sterilization temperature at which the medical supply is sterilized.

6. The assembly of claim 5, wherein the first temperature threshold is greater than the sterilization temperature.

7. The assembly of claim 6, wherein the sterilization temperature is about 49° C. (about 120° F.), wherein the first temperature threshold is about 66° C. (about 150° F.), and wherein the sealing temperature is about 138° C. (about 280° F.).

8. The assembly of claim 1, wherein the thermal pattern comprises one or more of a thermally sensitive dye, a thermally sensitive pigment, or a thermally sensitive ink.

9. The assembly of claim 1, wherein the thermal pattern comprises one or more of a substantially continuous line, a substantially continuous band, a logo, a word, or a phrase, a motif, or visual indicia, or a repeating pattern of the logo, the word, or the phrase.

10. The assembly of claim 1, wherein a difference between the first, second, and third visible indicia is visually perceptible under one or both of visible light or ultraviolet light.

11. The assembly of claim 1, wherein the medical package comprises one or more of a woven panel, a non-woven panel, a plastic panel, a polymer film, a paper film, a coated paper film, a metalized polymer film, a foil, a tray, a thermoform, a two-piece clamshell, or a folded clamshell.

12. The assembly of claim 1, further comprising the medical supply enclosed within the housing.

13. The assembly of claim 1, wherein the thermal pattern comprises a first thermal pattern defined by a first thermal composition and a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

14. An article comprising:
a first package portion comprising a first peripheral region;
a second package portion comprising a second peripheral region substantially aligned with the first peripheral region; and
a medical supply secured between the first and second package portions, wherein at least one of the first or second peripheral regions comprises a thermal pattern configured to:
exhibit a first visible indicium indicative of an unsealed region between the first and second peripheral regions or an unsterilized state of the medical package,
irreversibly transform from the first visible indicium to a second visible indicium indicative of a sealed region between the first and second peripheral regions in response to exposure to a temperature greater than or equal to a first temperature threshold, and
irreversibly transform from the first visible indicium to a third visible indicium indicative of a sterilized state of the medical package in response to exposure to a temperature greater than or equal to a second temperature threshold, wherein at least a portion of the third visible indicium is spaced from the first and second sealable regions.

15. The article of claim 14, wherein the first temperature threshold is less than or equal to a sealing temperature at which the first and second peripheral regions seal together, and wherein the second temperature threshold is less than or equal to a sterilization temperature at which the medical supply is sterilized.

16. The article of claim 15, wherein the first temperature threshold is greater than the sterilization temperature.

17. The article of claim 14, wherein the thermal pattern comprises one or more of a thermally sensitive dye, a thermally sensitive pigment, or a thermally sensitive ink.

18. The article of claim 14, wherein the thermal pattern comprises one or more of a substantially continuous line, a substantially continuous band, a logo, a word, or a phrase, a repeating pattern of the logo, the word, or the phrase, a motif, or visual indicia.

19. The article of claim 14, wherein a difference between the first, second, and third colors or patterns is visually perceptible under one or both of visible light or ultraviolet light.

20. The article of claim 14, wherein one or both of the first and second package portion comprises one or more of a woven panel, a non-woven panel, a plastic panel, a polymer film, a paper film, a coated paper film, a metalized polymer film, a foil, a tray, a thermoform, a two-piece clamshell, or a folded clamshell.

21. The article of claim 14, wherein the thermal pattern comprises a first thermal pattern defined by a first thermal composition and a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

22. A method comprising:
aligning a first sealable region of a medical package and a second sealable region of the medical package, wherein the medical package defines a housing configured to receive a medical supply; and
heating at least one of the first or second sealable regions to a sealing temperature to seal the first and second sealable regions together,
wherein the medical package comprises a thermal pattern configured to:
exhibit a first visible indicium indicative of an unsealed region between the first and second sealable regions or an unsterilized state of the medical package,
irreversibly transform from the first visible indicium to a second visible indicium indicative of a sealed region between the first and second sealable regions in response to exposure to a temperature greater than or equal to a first temperature threshold, and
irreversibly transform from the first visible indicium to a third visible indicium indicative of a sterilized state of the medical package in response to exposure to a temperature greater than or equal to a second temperature threshold, wherein at least a portion of the third visible indicium is spaced from the first and second sealable regions.

23. The method of claim 22, wherein aligning the first sealable region of the medical package and the second sealable region of the medical package comprises aligning a first package portion with a second package portion, the housing being defined between the first and second package portions.

24. The method of claim 22, wherein heating the at least one of the first or second sealable regions to the sealing temperature seals the first and second sealable regions together to substantially close the housing.

25. The method of claim 22, wherein the first and second sealable regions are configured to be sealed together to form a sterile barrier to the housing.

26. The method of claim 22, wherein heating the at least one of the first and sealable regions to the sealing temperature comprises impulse sealing the first and second sealable regions.

27. The method of claim 22, further comprising, before heating the at least one of the first and sealable regions to the sealing temperature, applying the thermal pattern to at least one of the first or second sealable regions and to the surface region.

28. The method of claim 27, wherein applying the thermal pattern comprises one or more of coating, stenciling, stamping, printing, or pad printing a thermal composition in a predetermined pattern.

29. The method of claim 27, wherein applying the thermal pattern comprises applying a first thermal pattern defined by a first thermal composition and applying a second thermal pattern defined by a second thermal composition, wherein the first thermal pattern is configured to transform to the second visible indicium in response to exposure to the temperature greater than or equal to the first temperature threshold, and wherein the second thermal pattern is configured to transform to the third visible indicium in response to exposure to the temperature greater than or equal to the second temperature threshold.

30. The method of claim 22, wherein the first temperature threshold is less than or equal to the sealing temperature, wherein the second temperature threshold is less than or equal to a sterilization temperature at which the medical supply is sterilized, and wherein the first temperature threshold is greater than or equal to the sterilization temperature.

31. The method of claim 22, further comprising,
introducing a medical supply into the housing; and
sterilizing the medical supply by at least exposing the medical supply and the medical package to a sterilization temperature for a predetermined duration of time, wherein the sterilization temperature is less than the first temperature threshold.

32. An assembly comprising:
a medical package defining a housing configured to receive a medical supply, the medical package comprising a first sealable region and a second sealable region, wherein the first and second sealable regions are configured to be sealed together to secure the medical supply within the housing,
wherein the medical package comprises:
a first thermal pattern defined by a first thermal composition configured to exhibit a first visible indicium indicative of an unsealed region between the first and second sealable regions and irreversibly transform to a second visible indicium indicative of a sealed region between the first and second sealable regions in response to exposure to a temperature greater than or equal to a first temperature threshold, and
a second thermal pattern defined by a second thermal composition configured to irreversibly transform to a third visible indicium indicative of a sterilized state of the medical package from a fourth visible indicium indicative of an unsterilized state of the medical package in response to exposure to a temperature greater than or equal to a second temperature threshold.

33. The assembly of claim 32, wherein at least a portion of the second thermal pattern is spaced from the first thermal pattern.

34. The assembly of claim 33, wherein the second thermal pattern is spaced in its entirety from the first thermal pattern.

35. The assembly of claim 32, wherein at least a portion of the second thermal pattern is spaced from the first and second sealable regions.

36. The assembly of claim 35, wherein the second thermal pattern is spaced in its entirety from the first and second sealable regions.

37. The assembly of claim 32, wherein the first temperature threshold is less than or equal to a sealing temperature at which the first and second sealable regions seal together, and wherein the second temperature threshold is less than or equal to a sterilization temperature at which the medical supply is sterilized.

38. The assembly of claim 37, wherein the first temperature threshold is greater than the sterilization temperature.

39. The assembly of claim 38, wherein the sterilization temperature is about 49° C. (about 120° F.), wherein the first temperature threshold is about 66° C. (about 150° F.), and wherein the sealing temperature is about 138° C. (about 280° F.).
